(19) United States
(12) Patent Application Publication
(10) Pub. No.: US 2010/0049292 A1
(43) Pub. Date: Feb. 25, 2010

(54) BALLOON FOR A BODY LUMEN AND METHOD OF USE

(75) Inventor: DAVID J. FIORELLA, Old Field, NY (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 12/435,423

(22) Filed: May 5, 2009

Related U.S. Application Data
(60) Provisional application No. 61/050,710, filed on May 6, 2008.

(51) Int. Cl. A61M 29/02; A61F 2/84

(52) U.S. Cl. 623/1.11; 606/194

(57) ABSTRACT

A balloon for use in a body lumen comprises a first inflatable balloon segment and a second inflatable balloon segment. The balloon has at least one opening for receiving inflation fluid to inflate the balloon segments. The first balloon segment when inflated includes a surface that defines a recess to receive at least a portion of the second balloon segment. The first and second balloon segments are arranged relative to each other such that a major portion of one of the first and second balloon segments is received in the body lumen before the other of the first and second balloon segments begins to be received in the body lumen.

### RELATED APPLICATION

This application claims priority from U.S. Provisional Application No. 61/050,710, filed May 6, 2008, the subject matter of which is incorporated herein by reference.

### TECHNICAL FIELD

The present invention relates to an apparatus for use in a body lumen and a method for such use and, more particularly, to a balloon for use in a body lumen and a method for such use.

### BACKGROUND OF THE INVENTION

Angioplasty is a medical procedure commonly used to address medical problems associated with narrowed blood vessels. Angioplasty involves inserting a balloon catheter into a patient's body and advancing the catheter into an artery. The balloon can be positioned in the artery adjacent to the site of the narrowing or stenosis and inflated to dilate or increase the internal diameter of the narrowed region. The balloon catheter can then be withdrawn.

In some cases, an artery with a stenosis that has been dilated by angioplasty can rebound or re-close over time, narrowing the artery again. An intraluminal prosthesis, such as a stent, can be used to help prevent the dilated region of an artery from narrowing after angioplasty. The intraluminal prosthesis, which typically has a tubular shape, can be placed in the dilated region of the artery to help maintain the increased internal diameter of the artery and help keep the lumen of the artery open. The prosthesis can be delivered to the required site with a balloon catheter.

### SUMMARY OF THE INVENTION

In an embodiment of the present invention, a balloon for use in a body lumen comprises a first inflatable balloon segment and a second inflatable balloon segment. The balloon has at least one opening for receiving inflation fluid to inflate the balloon segments. The first balloon segment when inflated includes a surface that defines a recess to receive at least a portion of the second balloon segment. The first and second balloon segments are arranged relative to each other such that a major portion of one of the first and second balloon segments is received in the body lumen before the other of the first and second balloon segments begins to be received in the body lumen.

In another embodiment of the invention, an apparatus is provided for insertion into a body lumen. The apparatus comprises a catheter, a first inflatable balloon element coupled to the catheter, and a second inflatable balloon element coupled to the catheter. The first balloon element when inflated includes a surface that defines a recess to receive at least a portion of the second balloon element. The first and second balloon elements are arranged sequentially relative to each other along the catheter.

In another embodiment of the invention, a method is provided for exerting a radially outward force on a body lumen. The method comprises providing a balloon catheter including first and second inflatable balloon elements. The first balloon element when inflated includes a surface that defines a recess to receive at least a portion of the second balloon element. The first and second balloon elements are arranged sequentially relative to each other along the catheter. The method also comprises inserting the balloon catheter into the body lumen and positioning the balloon catheter within the body lumen such that at least one of the first and second balloon elements is located radially adjacent the stenosis. The method further comprises expanding the at least one of the first and second balloon elements so as to exert a radially outward force on body lumen.

### BRIEF DESCRIPTION OF THE DRAWINGS

For a better understanding of the invention, reference may be made to the accompanying drawings, in which:

FIG. 1 is a side view, partly in section, of one embodiment of the present invention in a body lumen;

FIG. 2 is a view similar to FIG. 1 in which a balloon of the embodiment of FIG. 1 has been inflated in the body lumen;

FIG. 3 is a sectional view corresponding to FIG. 2;

FIG. 4 is an enlarged view of a portion of FIG. 3;

FIG. 5 is a side view of a second embodiment of the present invention; and

FIG. 6 is an enlarged sectional view of a portion of FIG. 5.

### DESCRIPTION OF EMBODIMENTS

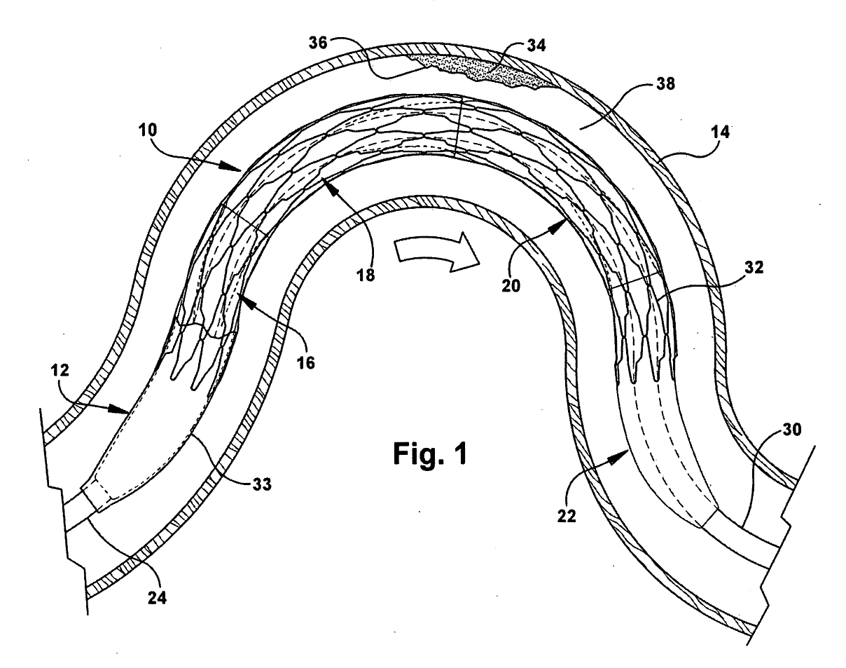

FIGS. 1 through 4 depict an apparatus 10 for use in a body lumen in accordance with a first example of the present invention. The apparatus 10 comprises a balloon catheter 12, which is illustrated as being located in a tortuous blood vessel 14. The balloon catheter 12 includes four balloon elements 16, 18, 20 and 22 arranged in series along a tube or catheter 24. As shown in FIG. 4, the tube 24 defines a lumen 25 that receives a smaller diameter sleeve 26. The sleeve 26, in turn, defines a lumen 27 that receives a flexible guide wire 28. The tube 24 has a distal end portion 30, which is partially shown in FIGS. 1-3. The distal end portion 30 of the tube 24 is introduced into the blood vessel 14 or other body lumen before other portions of the tube. The balloon element 22 is located closer to the distal end portion 30 of the tube 24 than the other balloon elements 16-20.

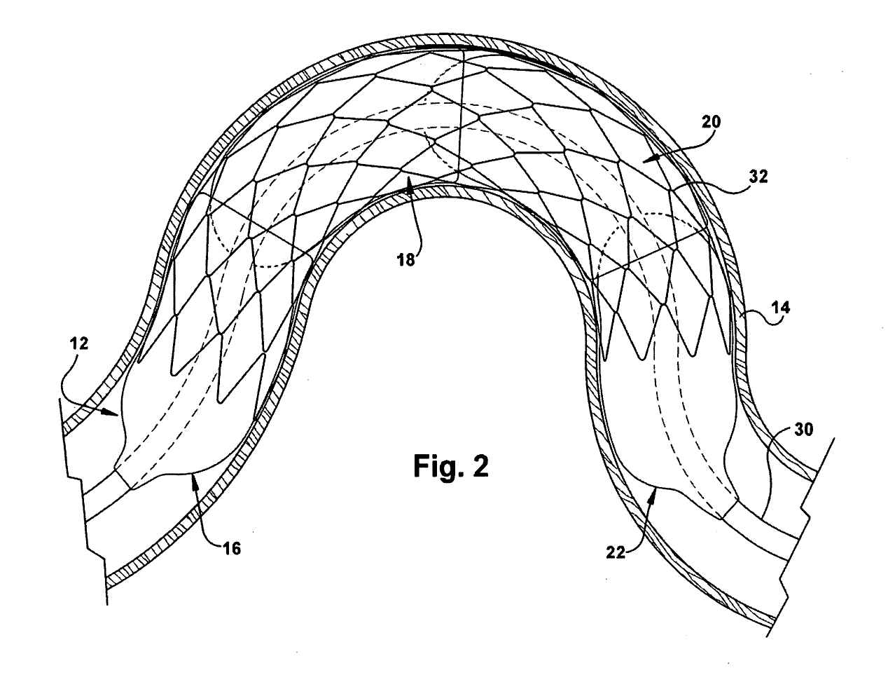

The balloon catheter 12 carries a stent 32. The stent 32 may contact the balloon elements 16-22 or the balloon elements may be covered by an optional sheath 33, which is partially shown in FIG. 1. The balloon catheter 12 and the stent 32, as illustrated in FIG. 1, are positioned radially and longitudinally adjacent a body of plaque 34 on the inner wall surface of the blood vessel 14, which has created a stenosis 36 or narrowing of the lumen 38 defined by the blood vessel. The balloon elements 16-22 of the balloon catheter 12 are inflatable, as will be described in greater detail below and as illustrated in FIG. 2, to apply a radially outward force to compress the body of plaque 34 and expand the stent 32 against the body of plaque to help maintain it in a compressed condition. Inflation of the balloon elements 16-22 is thus effective to dilate the stenosis 36 and return the lumen 38 to substantially its original diameter.

To facilitate (a) introducing the balloon catheter 12 into the tortuous blood vessel 14 or a tortuous lumen of another body organ and (b) maneuvering the balloon catheter within blood vessel or other organ, the balloon elements 16-22 are configured to allow relative movement between adjacent balloon elements. The configuration of the balloon elements 16-22 can be explained with particular reference to adjacent balloon elements 20 and 22, which are representative of the configuration of the other adjacent balloon elements.

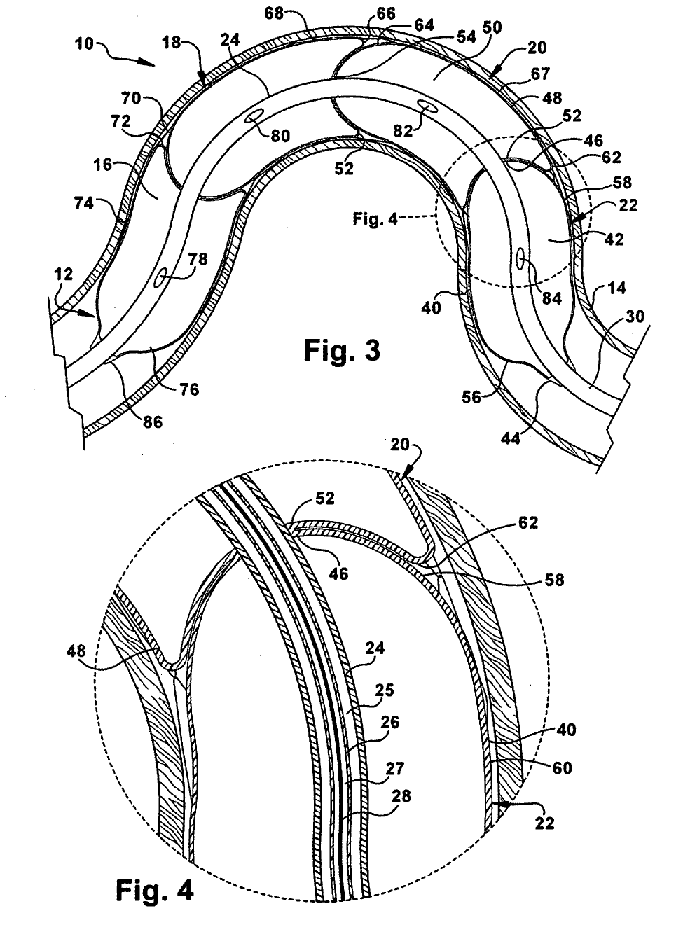

Balloon element 22 comprises a generally tubular wall 40 that, together with a portion of the tube 24, encloses an inflatable volume 42. Opposite right and left ends 44 and 46, as viewed in FIGS. 3 and 4, of the tubular wall 40 fit closely about the tube 24 to help provide a relatively fluid tight seal to permit inflation of the inflatable volume 42 by an inflation fluid. Adjacent balloon element 20 similarly comprises a generally tubular wall 48 that, together with a portion of the tube 24, encloses an inflatable volume 50. Opposite right and left ends 52 and 54, as viewed in FIGS. 3 and 4, of the tubular wall 48 fit tightly about the outer surface of the tube 24 and may be otherwise sealed to provide a substantially fluid tight seal to permit inflation of the inflatable volume 50 by an inflation fluid.

The rightward end surface 56 of the wall 40 of balloon element 22, as viewed in FIGS. 3 and 4, has a tapered shape. The rightward end surface 56 meets the tube 24 at the right end 44 of the wall 40 adjacent the distal end portion 30 of the tube and tapers radially outward in a direction away from the distal end portion of the tube. The tapered shape of the end surface 56 facilitates introducing the balloon element 22 into the blood vessel 14 and maneuvering the balloon element past and through obstructions, such as the body of plaque 34.

The opposite or leftward end surface 58 of the wall 40 of the balloon element 22 is closely adjacent to the tube 24 at the left end 46 of the wall, which is a location on the balloon element 22 farthest from the distal end portion 30 of the tube. The leftward end surface 58, when the balloon element 22 is inflated, then curves radially outward in a direction toward the distal end portion 30 of the tube 24. The curvature of the end surface 58 provides an overall convex shape to the end surface. An intermediate surface 60 of the wall 40 of the balloon element 22 extends between the leftward and rightward end surfaces 58 and 56.

Adjacent the leftward end surface 58 of the wall 40 of the balloon element 22 is a rightward end surface 62 of the wall 48 of the adjacent balloon element 20. The rightward end surface 62 is closely adjacent to the tube 24 at the right end 52 of the wall 48 and adjoins the leftward end surface 58 at the left end 46 of the wall 40 of the balloon element 22. The rightward end surface 62, when the balloon element 20 is inflated, curves radially outward from the right end 52 of the wall 48 in a direction toward the distal end portion 30 of the tube 24, which provides an overall concave shape to the end surface 62. The concave curvature of the rightward end surface 62 of the balloon element 20, when inflated, provides a shape that generally conforms to the convex shape of the adjacent leftward end surface 58 of the balloon element 22, when inflated. The concave and convex shapes of the end surfaces 62 and 58, respectively, also effectively provides a “ball-and-socket” joint or interface between the adjacent balloon elements 20 and 22. The conforming shapes of the end surfaces 62 and 58 further mean that the end surface 62 defines a recess that at least partially receives the end surface 58, and the balloon element 20 at least partially overlaps the balloon element 22. The overlap between the balloon elements 20 and 22 preferably occurs both when the balloon elements are inflated and when they are collapsed.

The leftward end surface 64 (FIG. 3) of the balloon element 20 is closely adjacent to the tube 24 at the left end 54 of the wall 48 of the balloon element, which is a location on the balloon element 20 that is farthest from the distal end portion 30 of the tube. The leftward end surface 64, when the balloon element 20 is inflated, then curves radially outward in a direction toward the distal end portion 30 of the tube 24, which provides an overall convex shape to the end surface. An intermediate surface 67 of the wall 48 of the balloon element 20 extends between the leftward and rightward end surfaces 64 and 62.

The conforming shapes of the end surfaces 58 and 62 of the balloon elements 22 and 20, respectively, are representative of the shapes of adjacent end surfaces 64 and 66 of walls 48 and 68 of the balloon elements 20 and 18, respectively, and adjacent end surfaces 70 and 72 of walls 68 and 74 of the balloon elements 18 and 16, respectively. Leftward end surface 76 of the wall 74 of the balloon element 16 has a shape that is generally a mirror image of the shape of the rightward end surface 56 of the balloon element 22. This tapered shape of the end surface 76 facilitates removing the balloon element 16 from the blood vessel 14 and maneuvering the balloon element past obstructions.

As shown in FIGS. 3 and 4, the balloon elements 16-22 are joined to one another at their respective adjacent ends near the tube 24. Thus, the illustrated balloon elements 16-22 are, in effect, segments of a single balloon. Nonetheless, other constructions of the balloon elements 16-22 are possible. For example, the balloon elements 16-22 could be formed as separate small balloons that are individually attached or coupled to the tube 24. As another alternative, the balloon elements 16-22 could be joined to one another by short tubular lengths of material such that the balloon elements are spaced a small distance from one another. The distance between the balloon elements 16-22 with such a construction must not be so large, however, as to interfere substantially with the benefits of having the conforming shapes of adjacent end surfaces of the balloon elements 16-22.

The balloon elements 16-22 have a collapsed condition, which is shown in FIG. 1, and an inflated condition, which is shown in FIGS. 2-4. The collapsed condition of the balloon elements 16-22 facilitates introducing the balloon catheter 12 into the blood vessel 14 or another body organ with a lumen and maneuvering the balloon catheter within the blood vessel or other organ so that the balloon catheter is properly positioned at a designated site. The inflated condition of the balloon elements 16-22 facilitates applying a radially outward force at the designated site to, for example, compress the body of plaque 34 and expand the stent 32.

The requirement for the balloon elements 16-22 to assume both a collapsed condition and an inflated condition, while also having adjacent surfaces of the balloon elements assume or maintain conforming shapes, means that the material of which the balloon elements are formed must have proper compliance or flexibility. A suitable material is a biocompatible plastic, such as polyurethane (PU), polyvinylchloride (PVC), polyethylene (PE), polyolefin co-polymer (POC) and/or polyethylene terephyhalate (PET), which can provide a generally uniform compliance throughout the wall of each balloon element 16-22. The balloon elements 16-22 may, however, have a compliance or flexibility that varies along the length of the wall of each balloon element. For example, the walls 74, 68, 48 and 40 of the balloon elements 16, 18, 20 and 22, respectively, may have a greater flexibility or compliance along the adjacent end surfaces 72, 70, 66, 64, 62 and 58 of the balloon elements and a lesser flexibility or compliance intermediate the end surfaces of the balloon elements. Conversely, the walls 74, 68, 48 and 40 of the balloon elements 16, 18, 20 and 22, respectively, may have a lesser flexibility or compliance along the adjacent end surfaces 72, 70, 66, 64, 62 and 58 of the balloon elements and a greater flexibility or compliance intermediate the end surfaces of the balloon elements. A variable compliance may be achieved, for example, via variations in the thickness of the walls 74, 68, 48 and 40 of the balloon elements 16-22 or via variations in the material of which the balloon elements are made.

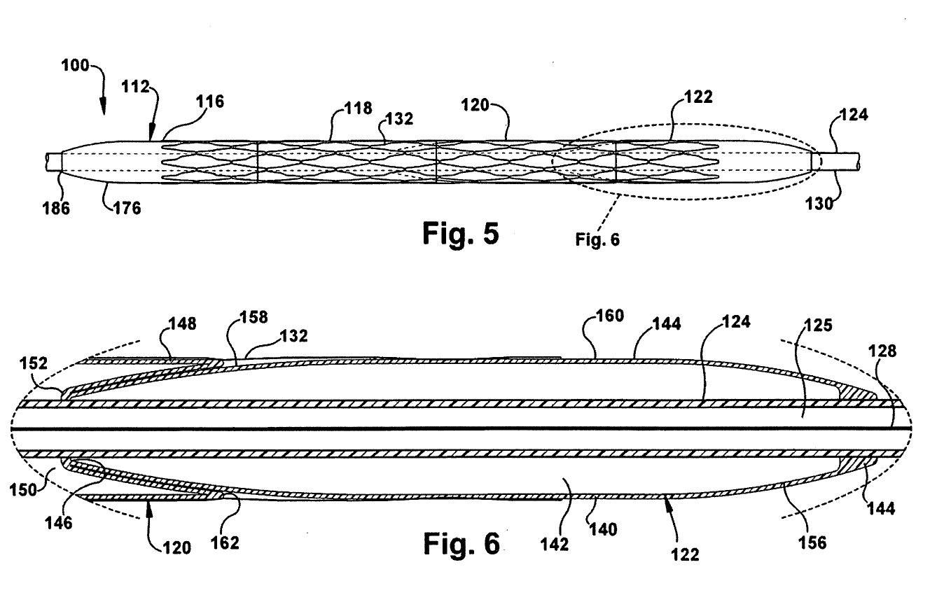

To facilitate achieving the collapsed condition of the balloon elements 16-22, it may be necessary to adjust the concave and convex shapes of the adjacent end surfaces 72, 70, 66, 64, 62 and 58 of the balloon elements. Specifically, the shapes of the adjacent end surfaces 72, 70, 66, 64, 62 and 58 are preferably spherical, when the balloon elements 16-22 are inflated, to provide a range of relative movement between adjacent balloon elements most closely resembling the movement provided by a ball-and-socket joint. To achieve such a spherical shape, however, the walls 74, 68, 48 and 40 of the balloon elements 16-22 may need to have a reduced compliance or flexibility along the adjacent end surfaces 72, 70, 66, 64, 62 and 58. Such reduced compliance may, in turn, reduce the ability of the walls 74, 68, 48 and 40 to fit close to another and to the tube 24 when the balloon elements 16-22 are in the collapsed condition, thereby resulting in an outer diameter of the series of collapsed balloon elements that is larger than desired. To achieve a smaller outer diameter of the series of balloon elements 16-22, in their collapsed condition, the preferred spherical shape of the end surfaces 72, 70, 66, 64, 62 and 58, when the balloon elements are inflated, may need to be adjusted to a shape more closely resembling a cone. One such shape is shown in FIGS. 5 and 6 and is described in greater detail below.

To inflate the balloon elements 16-22, an inflation fluid must be introduced into the balloon elements. Inflation fluid, such as air or an inert gas, is delivered to the balloon elements 16-22 of the balloon catheter 12 from a syringe (not shown) or other pumping element located outside of a patient's body. In the embodiment of FIGS. 1-4, inflation fluid is delivered via the lumen 25 defined by the tube 24 and outside of the sleeve 26. To allow the inflation fluid to enter each balloon element 16-22, fluid flow openings 78, 80, 82 and 84 are formed in the tube 24 inside the balloon elements 16, 18, 20 and 22, respectively. Although only one fluid flow opening 78, 80, 82 and 84 is formed in the tube 24 inside each balloon element 16, 18, 20 and 22, respectively, in the embodiment of FIGS. 1-4, two or more fluid flow openings may alternatively be formed in the tube inside each balloon element. The dimensions of the fluid flow openings 78, 80, 82 and 84 may be determined by, among other things, the volumes of the respective balloon elements 16-22, the extent to which each balloon element is to be inflated, the volume of inflation fluid flowing through the lumen 25, and the pressure of the inflation fluid.

As another alternative, the flow area of the fluid flow opening 78 inside the balloon element 16 may be smaller than the flow area of the fluid flow opening 80 inside the balloon element 18, which, in turn, may be smaller than the flow area of the fluid flow opening 82 inside the balloon element 20. The flow area of the fluid flow opening 82 may then be smaller than the flow area of the fluid flow opening 84 inside the balloon element 22. Providing different relative sizes for the fluid flow openings 78, 80, 82 and 84 or providing different numbers of fluid flow openings in each balloon element 16, 18, 20, and 22 may help compensate for the loss of inflation fluid in the lumen 25 as a portion of the inflation fluid flows into each of the balloon elements.

Alternative structures can be used to deliver inflation fluid to each of the balloon elements 16-22. For example, within the tube 24, there may be longitudinally extending dividing walls (not shown) that divide the lumen 25 into a series of adjacent smaller lumens, one to receive the guide wire 28 and four additional smaller lumens to deliver inflation fluid to each of the balloon elements 16-22. Such an arrangement would allow inflation fluid flow to be controlled dynamically at the time of inflation of the balloon elements 16-22 by controlling inflation fluid flow through each of the smaller lumens individually, rather than relying on a fixed size of the fluid flow openings 78, 80, 82 and 84. Such arrangement may, however, require more space in the balloon catheter 12 in order to accommodate the dividing walls.

As another alternative, the sleeve 26 could be eliminated, and the balloon catheter 12 would include just the tube 24. The tube 24 would then function both to receive the guide wire 28 and to deliver inflation fluid to the balloon elements 16-22. As a further alternative, the tube 24 could terminate adjacent the leftward end surface 76 of the wall 74 of the balloon element 16. The left end 86 of the wall 74 would fit tightly about the tube 24 and might otherwise be sealed to the tube to provide an effective seal against leakage of inflation fluid from the balloon element 16. The walls 74, 68, 48 and 40 of the balloon elements 16, 18, 20 and 22, respectively, would not fit closely about the sleeve 26 for the guide wire 28, with one exception, but would instead remain spaced away from the sleeve to provide a series of passages for inflation fluid to flow sequentially from one balloon element to the next. Such an inflation fluid flow from one balloon element to the next may facilitate inflation of all of the balloon elements 16, 18, 20 and 22. Only the rightward end 44 of the wall 40 of the balloon element 22 would fit tightly about the sleeve 26 to provide an effective seal against leakage of inflation fluid from the balloon element 22.

Regardless of the structure used to deliver inflation fluid to the balloon elements 16-22, the right end 44 of the wall 40 and the left end 86 of the wall 74 should fit tightly about the tube 24 or other, similarly positioned element of the balloon catheter 12 to provide an effective seal against leakage of inflation fluid from the balloon elements. In addition, the rightward end 44 of the wall 40 of the balloon element 22 should fit tightly about the tube 24 or other, similarly positioned element of the balloon catheter 12 to ensure that the balloon elements 16-22 move along with the tube 24 to the intended site in the blood vessel 14 or other body organ with a lumen. The tight fit couples the balloon elements 16-22 to the tube 24, which may be made of any suitable biocompatible plastic material. Similarly, the distal end portion 30 of the tube 24 should fit tightly about the sleeve 26, which may be made of any suitable biocompatible plastic material, to ensure that the balloon elements 16-22 and the tube move along with the sleeve 26 to the intended site, together with the guide wire 28. The tight fit and substantially fluid tight seal can be provided, in each case, via a friction or interference fit, friction sleeves, heat-shrink tubing, biocompatible cements or other adhesives, or tapered or variable cross-sections for the walls 74 and 40 of the balloon elements 16 and 22, respectively, and/or the walls of the tube 24 and sleeve 26.

In the embodiment of the invention illustrated in FIGS. 1-4, not only does the rightward end 44 of the balloon element 22 fit tightly about the tube 24, the distal end portion 30 of the tube fits tightly about the sleeve 26 and the sleeve fits tightly about the guide wire 28. Thus, the guide wire 28 brings with it the sleeve 26, the sleeve brings with it the tube 24, and the tube brings with it the balloon elements 16-22.

In use, the balloon catheter 12 is first positioned adjacent a location at which it will be introduced into a patient's body. If the balloon catheter 12 is to be introduced into a blood vessel 14, an incision or puncture (not shown) must be made to permit access to the blood vessel. If the balloon catheter 12 is to be introduced into another body organ with a lumen, it may be possible to position the balloon catheter at a naturally occurring opening in the body, such as the entrance to the urethra. To introduce the balloon catheter into the blood vessel 14, a distal end (not shown) of the guide wire 28 is first introduced into the lumen 38 defined by the blood vessel, followed by the distal end portion 30 of the tube 24. The end surface 56 of the wall 40 of the balloon element 22, in its collapsed condition, is then introduced into the lumen 38. As can been seen from FIG. 1, a major portion of the balloon element 22 will be received in the lumen 38 before the next balloon element 20 is introduced into the lumen.

Each balloon element 22, 20, 18 and 16 is successively introduced into the lumen 38 in the manner just described in the direction of the arrow in FIG. 1. The guide wire 28 is then maneuvered from outside the patient's body to bring one or more of the balloon elements to a designated site in the patient's body, such as the location of the body of plaque 34 illustrated in FIG. 1. Although the designated site may be in the blood vessel 14 into which the balloon catheter 12 is first introduced, it may be necessary to maneuver the guide wire 28 through multiple blood vessels and/or other body organs with lumens to reach the designated site.

To reach the designated site in the patient's body, the balloon catheter may have to pass through portions of the blood vessel 14 that are tortuous in shape and/or small in diameter. The collapsed condition of the balloon elements 16-22 will facilitate such passage, as will the conforming shapes of the adjacent end surfaces of the balloon elements. As can best be seen in FIG. 3, the adjacent end surfaces 58, 62, 64, 66, 70 and 72 of the balloon elements 22, 20, 18 and 16, when the balloon elements are inflated, effectively provide “ball-and-socket” joints or interfaces between the balloon elements. Such joints permit multi-directional flexibility of the series of balloon elements 16-22 as they pass through the tortuous lumen 38. The multi-directional flexibility includes relative rotational or pivotal movement between adjacent balloon elements 16-22 in upward and downward directions, as viewed in FIGS. 1-4 and also in directions into and out of the plane of FIGS. 1-4. To the extent the curved concave and convex shapes of the adjacent end surfaces 58, 62, 64, 66, 70 and 72 can be maintained when the balloon elements 16-22 are in their collapsed conditions, the multi-directional flexibility of the series of balloon elements will be similarly maintained.

At the designated site, the balloon elements 16-22 are maneuvered so that at least one balloon element is positioned radially and longitudinally adjacent the site. The balloon elements 16-22 are then inflated by inflation fluid delivered through the tube 24 from the proximal end (not shown) of the balloon catheter 12. By way of example, as can be seen in FIGS. 1 and 2, balloon elements 18 and 20 may positioned radially adjacent a body of plaque 34 on the inner wall surface of the blood vessel 14, which has created a stenosis 36 or narrowing of the lumen 38 defined by the blood vessel. When the balloon elements 16-22 of the balloon catheter 12 are inflated, the balloon elements 18 and 20 apply a radially outward force to compress the body of plaque 34. The inflation of the balloon elements 18 and 20 thus is effective to dilate the stenosis 36 and return the lumen 38 to substantially its original diameter. The sheath 33, which helps to reduce the possibility of the stent 32 rubbing against the balloon elements 16-22, will expand during inflation of the balloon elements 16-22.

In the embodiment of the invention illustrated in FIGS. 1-4, the balloon catheter 12 carries a stent 32. When the balloon catheter 12 is positioned radially adjacent the body of plaque 34 on the inner wall surface of the blood vessel 14, at least a portion of the stent is likewise positioned radially adjacent the body of plaque 34. Inflation of the balloon elements 16-22 applies a radially outward force to expand the stent 32 against the body of plaque 34 to help maintain it in a compressed condition.

As can be seen in FIGS. 2 and 3, the conforming shapes of the adjacent end surfaces 58, 62, 64, 66, 70 and 72 of the balloon elements 22, 20, 18 and 16, when the balloon elements are inflated, permits a generally continuous and substantially uniform outward pressure to be applied by the balloon elements. Specifically, because balloon element 18, for example, provides a concave recess in the end surface 66 of its wall 68, which receives the convex end surface 64 of the wall 48 of adjacent balloon element 20, the balloon element 18 overlaps a portion of the balloon element 20. The balloon element 18 thereby substantially fills the gap that would otherwise exist between the convexly curved end surface 64 of the balloon element 20, on the one hand, and the stent 32 and the wall of the blood vessel 14, on the other hand. The overlap of the balloon elements 18 and 20 provides good apposition of the stent 32 against the wall of the blood vessel 14.

The conforming shapes of the adjacent end surfaces 58, 62, 64, 66, 70 and 72 of the balloon elements 22, 20, 18 and 16, when the balloon elements are inflated, also permits the series of balloon elements to be inflated in an overall curved configuration to help maintain the normal or native tortuous configuration of the blood vessel 14 even at relatively high levels of inflation pressure. Further, the conforming shapes of the adjacent end surfaces 58, 62, 64, 66, 70 and 72 of the balloon elements 22, 20, 18 and 16, when the balloon elements are inflated, permit the stent 32 to be expanded or deployed in a curved configuration without significant distortion of the blood vessel 14.

After the balloon elements 16-22 of the balloon catheter 12 are inflated to compress the body of plaque 34 and expand the stent 32, the balloon elements are returned to their collapsed condition by permitting the inflation fluid to flow out through the proximal end (not shown) of the balloon catheter. The balloon catheter 12 is then withdrawn from the blood vessel 14 and the lumen 38, leaving the stent 32 in place. As previously noted, the tapered shape of the end surface 76 of the balloon element 16 facilitates removing the balloon elements 16-22 from the blood vessel 14 and maneuvering the balloon elements past obstructions.

FIGS. 5 and 6 illustrate a second embodiment 100 of an apparatus in accordance with the present invention. The apparatus 100 comprises a balloon catheter 112, which includes four balloon elements 116, 118, 120 and 122 arranged in series along a catheter or tube 124. As shown in FIG. 6, the tube 124 is tubular and defines a lumen 125 that receives a flexible guide wire 128. The tube 124 has a distal end portion 130, which is partially shown in FIG. 5. The distal end portion 130 of the tube 124 is introduced into a blood vessel (not shown) or other body lumen before other portions of the tube. The balloon element 122 is located closer to the distal end portion 130 of the tube 124 than the other balloon elements 116-120.

The balloon catheter 112 carries a stent 132. The balloon catheter 112 and the stent 132 can positioned radially and longitudinally adjacent a desired site inside a lumen (not shown) of a patient's body, such as adjacent a body of plaque on the inner wall of a blood vessel, which has created a stenosis or narrowing of the lumen defined by the blood vessel. The balloon elements 116-122 of the balloon catheter 112 are inflatable, as will be described in greater detail below, to apply a radially outward force to compress the body of plaque and expand the stent 132 against the body of plaque to help maintain it in a compressed condition. The inflation of the balloon elements 116-122 thus is effective to dilate the stenosis and return the lumen to substantially its original diameter.

To facilitate (a) introducing the balloon catheter 112 into a tortuous blood vessel or a tortuous lumen of another body organ and (b) maneuvering the balloon catheter within the blood vessel or other organ, the balloon elements 116-122 are configured to allow relative movement between adjacent balloon elements. The configuration of the balloon elements 116-122 can be explained with particular reference to adjacent balloon elements 120 and 122, which are representative of the configuration of the other adjacent balloon elements.

Balloon element 122 comprises a generally tubular wall 140 that, together with a portion of the tube 124, encloses an inflatable volume 142. Opposite right and left ends 144 and 146, as viewed in FIGS. 5 and 6, of the tubular wall 140 fit tightly about and may otherwise be sealed to the outer surface of the tube 124 to provide a substantially fluid tight seal to permit inflation of the inflatable volume 142 by an inflation fluid. Adjacent balloon element 120 similarly comprises a generally tubular wall 148 that, together with a portion of the tube 124, encloses an inflatable volume 150. Right end 152 and an opposite left end (not shown), as viewed in FIGS. 5 and 6, of the tubular wall 148 fit closely about the tube 124 to help provide a relatively fluid tight seal to permit inflation of the inflatable volume 150 by an inflation fluid.

The rightward end surface 156 of the wall 140 of balloon element 122, as viewed in FIGS. 5 and 6, has a tapered shape. The rightward end surface 156 meets the tube 124 at the right end 144 of the wall 140 adjacent the distal end portion 130 of the tube and tapers radially outward in a direction away from the distal end portion of the tube. The tapered shape of the end surface 156 facilitates introducing the balloon element 122 into a blood vessel and maneuvering the balloon element past and through obstructions.

The opposite or leftward end surface 158 of the wall 140 of the balloon element 122 is closely adjacent to the tube 124 at the left end 146 of the wall, which is a location on the balloon element 122 farthest from the distal end portion 130 of the tube. The leftward end surface 158, when the balloon element 122 is inflated, then angles radially outward in a direction toward the distal end portion 130 of the tube 124. An intermediate surface 160 of the wall 140 of the balloon element 122 extends between the leftward and rightward end surfaces 158 and 156.

Adjacent the leftward end surface 158 of the wall 140 of the balloon element 122 is a rightward end surface 162 of the wall 148 of the adjacent balloon element 120. The rightward end surface 162 is closely adjacent to the tube 124 at the right end 152 of the wall 148 and adjoins the leftward end surface 158 at the left end 146 of the wall 140 of the balloon element 122. The rightward end surface 162, when the balloon element 120 is inflated, angles radially outward from the right end 152 of the wall 148 in a direction toward the distal end portion 130 of the tube 124. The shape of the rightward end surface 162 of the balloon element 120, when inflated, generally conforms to the shape of the adjacent leftward end surface 158 of the balloon element 122, when inflated. The conforming shapes of the end surfaces 162 and 158 also mean that the end surface 162 defines a recess that at least partially receives the end surface 158, and the balloon element 120 at least partially overlaps the balloon element 122. The overlap between the balloon elements 120 and 122 preferably occurs both when the balloon elements are inflated and when they are collapsed.

The conforming shapes of the end surfaces 158 and 162 of the balloon elements 122 and 120, is representative of the shapes of adjacent end surfaces (not shown) of the balloon elements 120 and 118 and the adjacent end surfaces (not shown) of the balloon elements 118 and 116. Leftward end surface 176 of the balloon element 116 has a shape that is generally a mirror image of the shape of the rightward end surface 156 of the balloon element 122. This tapered shape of the end surface 176 facilitates removing the balloon element 116 from a blood vessel or other body organ with a lumen and maneuvering the balloon element past obstructions.

As shown in FIG. 6 with respect to balloon elements 120 and 122, the balloon elements 116-122 are joined to one another at their respective adjacent ends near the tube 124. Thus, the illustrated balloon elements 116-122 are, in effect, segments of a single balloon. Nonetheless, other constructions of the balloon elements 116-122 are possible. For example, the balloon elements 116-122 could be formed as separate small balloons that are individually attached or coupled to the tube 124. As another alternative, the balloon elements 116-122 could be joined to one another by short tubular lengths of material such that the balloon elements are spaced a small distance from one another. The distance between the balloon elements 116-122 with such a construction must not be so large, however, as to interfere substantially with the benefits of the conforming shapes of adjacent end surfaces of the balloon elements.

The balloon elements 116-122 have a collapsed condition, which is shown in FIGS. 5 and 6, and an inflated condition, which is not illustrated but which resembles the inflated condition of the embodiment shown in FIGS. 2-4. The collapsed condition of the balloon elements 116-122 facilitates introducing the balloon catheter 112 into a blood vessel (not shown) or other body organ with a lumen and maneuvering the balloon catheter within the blood vessel or other organ so that the balloon catheter is properly positioned at a designated site. The inflated condition of the balloon elements 116-122 facilitates applying a radially outward force at the designated site to, for example, compress a body of plaque and expand the stent 132.

The requirement for the balloon elements 116-122 to assume both a collapsed condition and an inflated condition, while also having adjacent surfaces of the balloon elements assume or maintain conforming shapes, means that the material of which the balloon elements are formed must have proper compliance or flexibility. A suitable material is a biocompatible plastic, such as polyurethane (PU), polyvinylchloride (PVC), polyethylene (PE), polyolefin co-polymer (POC) and/or polyethylene terephyhalate (PET), which can provide a generally uniform compliance throughout each balloon element 116-122. The balloon elements 116-122 may, however, have a compliance or flexibility that varies along the length of the wall of each balloon element. For example, the walls of the balloon elements 116-122 may have a greater flexibility or compliance along the adjacent end surfaces of the balloon elements and a lesser flexibility or compliance intermediate the end surfaces of the balloon elements. Conversely, the walls of the balloon elements 116-122 may have a lesser flexibility or compliance along the adjacent end surfaces of the balloon elements and a greater flexibility or compliance intermediate the end surfaces of the balloon elements. A variable compliance may be achieved, for example, via variations in the thickness of the walls of the balloon elements 116-122 or via variations in the material of which the balloon elements are made.

To inflate the balloon elements 116-122, an inflation fluid must be introduced into the balloon elements. Inflation fluid, such as a radiographic contrast material, an inert gas or air, is delivered to the balloon elements 116-122 of the balloon catheter 112 via a syringe (not shown) or other pumping element located outside of a patient's body. In the embodiment of FIGS. 5 and 6, inflation fluid is delivered via the lumen 125 defined by the tube 124. To allow the inflation fluid to enter each balloon element 116-122, fluid flow openings (not shown) are formed in the tube 124 inside the balloon elements 116-122. As previously described with respect to the embodiment of FIGS. 1-4, there may be only a single fluid flow opening in each balloon element 116-122 or multiple fluid flow openings in one or more balloon elements. The flow areas of the fluid flow openings may progressively increase in size from the fluid flow opening inside the balloon element 116 to the fluid flow opening inside the balloon element 122. The relative sizes of the fluid flow openings may help compensate for the loss of inflation fluid in the lumen 125 as a portion of the inflation fluid flows into each of the balloon elements 116-122. The dimensions of the fluid flow openings may be determined by, among other things, the volumes of the respective balloon elements 116-122, the extent to which each balloon element is to be inflated, the volume of inflation fluid flowing through the lumen 125, and the pressure of the inflation fluid.

Regardless of the structure used to deliver inflation fluid to the balloon elements 116-122, the ends 144 and 186 of balloon elements 122 and 116, respectively, should fit tightly about the tube 124 or other, similarly positioned element of the balloon catheter 112 to provide an effective seal against leakage of inflation fluid from the balloon elements. In addition, the rightward end 144 of the wall 140 of the balloon element 122 should fit tightly about the tube 124 or other, similarly positioned element of the balloon catheter 112 to ensure that the balloon elements 116-122 move along with the guide wire 128 to the intended site in the blood vessel or other body organ with a lumen. The tight fit couples the balloon elements 116-122 to the tube 124, which may be made of any suitable biocompatible plastic material, and which, together with the guide wire 128, defines a catheter. The tight fit can be provided, in each case, via a friction or interference fit, friction sleeves, heat-shrink tubing, biocompatible cements or other adhesives, or tapered or variable cross-sections to facilitate connection of the tube 124 and the walls of the balloon elements 116 and 122.

In the embodiment of the invention illustrated in FIGS. 5 and 6, not only does the rightward end 144 of the balloon element 122 fit tightly about the tube 124, the distal end portion 130 of the tube fits tightly about the guide wire 128. Thus, the guide wire 128 brings with it the tube 124, and the tube brings with it the balloon elements 116-122.

In use, the balloon catheter 112 is first positioned adjacent a location at which it will be introduced into a patient's body. If the balloon catheter 112 is to be introduced into a blood vessel, an incision or puncture (not shown) must be made to permit access to the blood vessel. If the balloon catheter 112 is to be introduced into another body organ with a lumen, it may be possible to position the balloon catheter at a naturally occurring opening in the body, such as the entrance to the urethra. To introduce the balloon catheter 112 into the blood vessel, a distal end (not shown) of the guide wire 128 is first introduced into the lumen defined by the blood vessel, followed by the distal end portion 130 of the tube 124. The end surface 156 of the balloon element 122, in its collapsed condition, is then introduced into the lumen. As can been seen from FIGS. 5 and 6, a major portion of the balloon element 122 will be received in the body lumen before the next balloon element 120 is introduced into the lumen.

Each balloon element 122, 120, 118 and 116 is successively introduced into the body lumen in the manner just described. The guide wire 128 is then maneuvered from outside the patient's body to bring one or more of the balloon elements to a designated site in the patient's body. Although the designated site may be in a blood vessel into which the balloon catheter 112 is first introduced, it may be necessary to maneuver the guide wire 128 through multiple blood vessels and/or other body organs with lumens to reach the designated site.

To reach the designated site in the patient's body, the balloon catheter may have to pass through portions of a blood vessel or other body lumen that are tortuous in shape and/or small in diameter. The collapsed condition of the balloon elements 116-122 will facilitate such passage, as will the conforming shapes of the adjacent end surfaces of the balloon elements. As can best be seen in FIG. 6, the adjacent end surfaces 158 and 162 of the balloon elements 122 and 120, respectively, when the balloon elements are collapsed and when they are inflated, effectively provide a flexible joint or interface between the balloon elements. Similar joints are formed between adjacent end surfaces of balloon element 120, 118 and 116. Such joints permit multi-directional flexibility of the series of balloon elements 116-122 through a tortuous lumen of a blood vessel or other body organ. The multi-directional flexibility includes relative rotational or pivotal movement between adjacent balloon elements 116-122 in upward and downward directions, as viewed in FIGS. 5 and 6, and also in directions into and out of the plane of FIGS. 5 and 6. As the shapes of the adjacent end surfaces can be maintained when the balloon elements 116-122 are in their collapsed conditions, the multi-directional flexibility of the balloon elements is also maintained in both conditions.

At the designated site, the balloon elements 116-122 are maneuvered so that at least one balloon element is positioned radially and longitudinally adjacent the site. The balloon elements 116-122 are then inflated by inflation fluid delivered through the tube 124 from the proximal end (not shown) of the balloon catheter 112. By way of example, balloon elements 116-120 may positioned radially adjacent a body of plaque on the inner wall of a blood vessel, which has created a stenosis or narrowing of the lumen defined by the blood vessel. When the balloon elements 116-122 of the balloon catheter 112 are inflated, the balloon elements apply a radially outward force to compress the body of plaque. The inflation of the balloon elements 116-122 thus is effective to dilate the stenosis and return the lumen to substantially its original diameter.

In the embodiment of the invention illustrated in FIGS. 5 and 6, the balloon catheter 112 carries a stent 132. When the balloon catheter 112 is positioned radially adjacent a body of plaque on the inner wall surface of a blood vessel, at least a portion of the stent is likewise positioned radially adjacent the body of plaque. Inflation of the balloon elements 116-122 applies a radially outward force to expand the stent 132 against the body of plaque to help maintain it in a compressed condition.

The conforming shapes of the adjacent end surfaces of the balloon elements 116-122, when the balloon elements are inflated, permits a generally continuous and substantially uniform outward pressure to be applied by the balloon elements. Specifically, because balloon element 120, for example, provides a recess in the end surface 162 of its wall 148, which receives the end surface 158 of the wall 148 of adjacent balloon element 122, the balloon element 120 overlaps a portion of the balloon element 122. The balloon element 120 thereby substantially fills the gap that would otherwise exist between the angled end surface 158 of the balloon element 122, on the one hand, and the stent 132 and the wall of a blood vessel (not shown), on the other hand. The overlap between the balloon elements 116-122 ensures good apposition of the stent 132 against the wall of a blood vessel or other body organ.

The conforming shapes of the adjacent end surfaces of the balloon elements 116-122, when the balloon elements are inflated, also permit the series of balloon elements to be inflated in an overall curved configuration to help maintain the normal or native tortuous configuration of a blood vessel even at relatively high levels of inflation pressure. Further, the conforming shapes of the adjacent end surfaces of the balloon elements 116-122, when the balloon elements are inflated, permit the stent 132 to be expanded or deployed in a curved configuration without significant distortion of a blood vessel.

After the balloon elements 116-122 of the balloon catheter 112 are inflated to compress a body of plaque and expand the stent 132, the balloon elements are returned to their collapsed condition by permitting the inflation fluid to flow out through the proximal end (not shown) of the balloon catheter. The balloon catheter 112 is then withdrawn from the blood vessel, leaving the stent 132 in place. As previously noted, the tapered shape of the end surface 176 of the balloon element 116 facilitates removing the balloon elements 116-122 from a blood vessel and maneuvering the balloon elements past obstructions.

While specific embodiments of the invention are shown in FIGS. 1-4 and in FIGS. 5 and 6 and described for use in dilation of a vascular stenosis and delivery of a balloon expandable stent, the invention may have other constructions and other uses. For example, although the embodiments of FIGS. 1-6 include a series of four balloon elements 16-22 and 116-122, the number of balloon elements can be as few as two and as many as ten or more. The sizes of the balloon elements 16-22 and 116-122 may vary depending upon the specific uses to which they are put and the lumens in which they are used. In general, for use in blood vessels, balloon elements 16-22 and 116-122 may have diameters in a range from about 1.5 millimeters to about 9 millimeters, and the overall length of a complete series of balloon elements 16-22 and 116-122 may be in a range from about 9 millimeter to about 40 millimeters. The balloon elements 16-22 and 116-122 may be inflated to different diameters or, potentially, somewhat different shapes than one another. Such balloon elements 16-22 and 116-122 of different diameters may be useful in expanding a stent, such as stent 32 and 132, to a non-uniform diameter along its length. For example, if the stent 32 and 132 is intended for placement at or adjacent a tapering (either inwardly or outwardly) or branching anatomical location, one or more successive balloon elements 16-22 and 116-122 could have differing diameters chosen to facilitate the desired differential expansion of the stent 32 and 132. These different diameters could be associated with different locations on a single tapered balloon element 16-22 and 116-122 and/or the different diameters could be associated with separate, substantially constant-diameter balloon elements 16-22 and 116-122. One or more of the balloon elements 16-22 and 116-122 may be made longer or shorter than other balloon elements, as another example, to adjust the flexibility of the series of balloon elements 16-22 and 116-122. In general, therefore, at least a portion of a chosen one of the first and second balloon elements 16-22 and 116-122 may have at least one of a different size, shape, material, thickness, permeability, conductivity, and resilience from a corresponding portion of an other one of the first and second balloon elements.

Alternative mechanisms can be used to deliver inflation fluid to each of the balloon elements 16-22 and 116-122. For example, within the tube 24 and 124, there may be longitudinally extending dividing walls that divide the lumen 25 and 125 into a series of adjacent smaller lumens, one to receive the guide wire 28 and 128 and additional smaller balloon inflation lumens (not shown) to deliver inflation fluid to each of the balloon elements 16-22 and 116-122. One of ordinary skill in the art could readily provide multiple balloon inflation lumens within the tube 24 and 124, with each balloon inflation lumen being associated with one or more balloon elements 16-22 and 116-122 for selective inflation of the associated balloon elements in a parallel manner, as opposed to the serial inflation provided by a common lumen 25 and 125. Such an arrangement would allow inflation fluid flow to be controlled dynamically at the time of inflation of the balloon elements 16-22 and 116-122 by controlling inflation fluid flow through each of the smaller lumens individually, rather than relying on fixed sizes of the fluid flow openings (not shown) to control inflation of the balloon elements 16-22 and 116-122. Such arrangement may, however, require more space in the catheter in order to accommodate the dividing walls.

It is contemplated that an apparatus 10 and 100 having multiple balloon inflation lumens could selectively inflate one or more balloon elements 16-22 and 116-122 in any desired sequence and at any desired timing during deployment of the apparatus. For example, the balloon elements 16-22 and 116-122 could be inflated asynchronously in proximal-to-distal order (left to right, in the orientation of the Figures), with each succeeding balloon element 16-22 and 116-122 being inflated once the previous balloon element 16-22 and 116-122 has reached a certain inflation percentage or level. (The term “asynchronously” is used herein to mean that two actions are not happening, existing, or arising at precisely the same time.) Indeed, in certain use applications, one or more balloon elements 16-22 and 116-122 may remain uninflated throughout use of the apparatus 10 and 100, as desired by a user. Additionally or alternatively, a manifold (not shown), such as, but not limited to, an automatically or manually controlled mechanical, pneumatic, hydraulic, or electric switch, may be provided to selectively inflate one or more of the balloon elements 16-22 and 116-122, either singly or in combination. For example, a mechanical shield arrangement could be placed in a user-accessible position at a proximal location on the multiple balloon inflation lumens to selectively block or crimp one or more of the multiple balloon inflation lumens in a predetermined sequence or pattern responsive to a user's twisting, pressing, releasing, or other manipulation of a shield controller, in order to provide asynchronous inflation of one or more of the balloon elements 16-22 and 116-122. As another example, the manifold may allow a user to sequentially fluidly connect a single source of inflation fluid to each of a group of multiple balloon inflation lumens, such as by use of a mechanism facilitating rotation of the single source into engagement with each of a group of circularly arranged balloon inflation lumens in turn for individual inflation of the balloon elements 16-22 and 116-122 in sequence using a single (and singly controlled) source of inflation fluid—this mechanism may operate analogously to the rotational firing of a Gatling gun.

Particularly when multiple balloon inflation lumens are provided to facilitate selective and discrete inflation of one or more of the balloon elements 16-22 and 116-122, the proximal-most and/or distal-most ones of the balloon elements 16, 22 and 116, 122 may be inflated before the other, remaining balloon elements 16-22 and 116-122. Such selective “anchor” pre-inflation may be helpful in preventing unwanted longitudinal migration of the balloon catheter 12 and 112 as the remaining balloon elements 18, 20 and 118, 120 are inflated to provide the desired therapeutic effect. Optionally, the pre-inflated proximal-most and/or distal-most balloon elements 16, 22 and 116, 122 may include a feature to enhance the anchoring function, such as, but not limited to, a friction coating and/or a thickened or stiffened wall (which also might allow inflation to a higher pressure than the inflation pressure of others of the balloon elements 16-22 and 116-122).

When one of the proximal-most or distal-most balloon elements 16, 22 and 116, 122 is pre-inflated to anchor the balloon catheter 12 and 112, the user may exert a slight longitudinal force upon the tube 24 and 124 to urge the remaining balloon elements 16-22 and 116-122 longitudinally away from the pre-inflated proximal-most or distal-most balloon elements 16, 22 and 116, 122. The pre-inflated proximal-most or distal-most balloon elements 16, 22 and 116, 122 will resist the longitudinal force to hold the balloon catheter 12 and 112 in the desired position within the body lumen, but the slight urging-away may help the remaining balloon elements 16-22 and 116-122 to efficiently inflate and achieve the conformed, “ball and socket”-type partially overlapping relationship described and shown herein.

As another inflation alternative, the balloon catheter 12 and 112 may include both the tube 24 and 124 and another tube or sleeve (not shown) smaller in diameter than the tube 24 and 124 and closely fitting the guide wire 28 and 128. The tube 24 and 124 would then function solely to deliver inflation fluid to the balloon elements 16-22 and 116-122. Although the tube 24 and 124 could continue to deliver inflation fluid through inflation fluid openings (not shown), the tube 24 and 124 could alternatively terminate adjacent the leftward end surface 76 and 176 of the balloon element 16 and 116. The left end 86 and 186 of the wall of the balloon element 16 and 116 would fit tightly about the tube 24 and 124 to provide an effective seal against leakage of inflation fluid from the balloon elements 16-22 and 116-122. The remainder of the walls of the balloon elements 16-22 and 116-122 would not fit closely about the smaller diameter tube (not shown) for the guide wire 28 and 128, with one exception, but would instead remain spaced away from the smaller diameter tube to provide a series of passages for inflation fluid to flow sequentially from one balloon element to the next. Such an inflation fluid flow from one balloon element to the next may facilitate inflation of all of the balloon elements 16-22 and 116-122. Only the rightward end 44 and 144 of the balloon element 22 and 122 would fit tightly about the smaller diameter tube (not shown) to provide an effective seal against leakage of inflation fluid from the balloon elements 16-22 and 116-122.

Although the balloon elements 16-20 and 116-120 have been shown as having recesses at their respective right ends to receive a portion of adjacent balloon elements, which should facilitate removal of the balloon catheters 12 and 112 from a body lumen, this arrangement may be reversed so that balloon elements 18-22 and 118-122 have recesses at their respective left ends to receive a portion of adjacent balloon elements. One or more of the balloon elements 16-22 and 116-122 may be covered with an outer tube or sleeve. Some portion of the balloon catheters 12 and 112 may be radiopaque to facilitate their placement, such as through use of fluoroscopy.

The stent 32 of FIGS. 1-4 and the stent 132 of FIGS. 5 and 6 are balloon-expandable stents but may be self-expanding stents. Each of the stents 32 and 132 may also be covered with an outer sheath, like the optional sheath 33 shown in FIG. 1, or lined with a layer of synthetic or biological material covering at least a portion of an inner or outer surface of the stent. Similarly, the stents 32 and 132 may be coated with a pharmaceutical or other therapeutic agent, which may be released, leached, diffused, or otherwise provided to a target tissue. Articulation points, such as joints or flex points in the structural pattern of stent material, may be provided in the stents 32 and 132. Such articulation points may be aligned with the interfaces or joints between adjacent balloon elements 16-22 and 116-122 and may thereby increase the overall flexibility of the stent and balloon catheter when used together.

The balloon catheters 12 and 112 are shown in FIGS. 1-6 and described as being for use in dilation of a vascular stenosis 36 but the balloon catheters may be used to dilate a stenosis in a lumen of any organ of the body, such as a ureter. The balloon catheters 12 and 112 are shown in FIGS. 1-6 and described as being for use in delivery of a balloon expandable stent 32 to maintain a vascular stenosis in a dilated condition, but the balloon catheters may be used to deliver a stent to a site of a vascular aneurysm or the site of a dilated stenosis in a lumen of any organ of the body, such as a ureter or kidney. Similarly, the balloon catheters 12 and 112 may be used to deliver the stents 32 and 132 to a site at which the stent spans segments of a blood vessel that either branches or changes significantly in diameter. In addition to the delivery of stents 32 and 132, the balloon catheters 12 and 112 could be used to deliver a graft, a patch, or the like, to a site in a blood vessel or any other organ of the body. Moreover, the balloon catheters 12 and 112 could be used without a stent 32 and 132, and with or without a sheath 33, in order to exert a radially outward force against a body lumen wall from inside the lumen as part of any medical procedure and for any reason; the articulation facilitated by the series of balloon elements 16-22 and 116-122 may help in maintaining or establishing a desired curvature in the body lumen while exerting the radially outward force in a substantially uniform manner longitudinally along the curved body lumen. For example, the balloon catheters 12 and 112 could assist in dilating a stricture in a tortuously curved portion of the small intestine without concurrently installing a stent 32 and 132.

From the above description of the invention, those skilled in the art will perceive improvements, changes and modifications. Such improvements, changes and modifications within the skill of the art are intended to be covered by the appended claims.

Having described the invention, the following is claimed:

1. A balloon for use in a body lumen, the balloon comprising a first inflatable balloon segment and a second inflatable balloon segment and having at least one opening for receiving inflation fluid to inflate the balloon segments, the first balloon segment when inflated including a surface that defines a recess to receive at least a portion of the second balloon segment, the first and second balloon segments being arranged relative to each other such that a major portion of one of the first and second balloon segments is received in the body lumen before the other of the first and second balloon segments begins to be received in the body lumen.

2. The balloon of claim 1 wherein the surface of the first balloon segment when inflated has a shape that generally conforms to a shape of an adjacent surface of the second balloon segment when inflated.

3. The balloon of claim 2 wherein the surface of the first balloon segment has a generally concave shape and the adjacent surface of the second balloon segment has a generally convex shape.

4. The balloon of claim 1 wherein the surface of the first balloon segment has a flexibility that is different than a flexibility of a portion of the first balloon segment that is spaced farther away from the second balloon segment.

5. The balloon of claim 1, configured for coupling to a catheter for insertion into the body lumen.

6. An apparatus for insertion into a body lumen, the apparatus comprising:
a catheter; and
a first inflatable balloon element coupled to the catheter and a second inflatable balloon element coupled to the catheter, the first balloon element when inflated including a surface that defines a recess to receive at least a portion of the second balloon element, the first and second balloon elements being arranged sequentially relative to each other along the catheter.

7. The apparatus of claim 6, wherein an expandable member is removably mounted on at least one of the first and second balloon elements for delivery to a site in the body lumen.

8. The apparatus of claim 6 wherein the expandable member includes at least one articulation point at which the expandable member can bend, the at least one articulation point being disposed radially adjacent an interface between the first and second balloon elements.

9. The apparatus of claim 6 wherein the surface of the first balloon element when inflated has a shape that generally conforms to a shape of an adjacent surface of the second balloon element when inflated.

10. The apparatus of claim 9 wherein the surface of the first balloon element has a generally concave shape and the adjacent surface of the second balloon element has a generally convex shape.

11. The apparatus of claim 6 wherein the catheter comprises at least one lumen for receiving a guide wire.

12. The apparatus of claim 6 wherein the catheter comprises at least one lumen for delivering inflation fluid to at least one of the first and second balloon elements.

13. The apparatus of claim 12 wherein the catheter comprises a plurality of lumens for delivering inflation fluid, each of the plurality of lumens delivering inflation fluid to at least one of the first and second balloon elements.

14. The apparatus of claim 13 wherein a chosen one of the plurality of lumens delivers inflation fluid to at least one of the first and second balloon elements asynchronously with the delivery of inflation fluid to at least one of the first and second balloon elements by an other one of the plurality of lumens.

15. The apparatus of claim 14 wherein a chosen one of the first and second balloon elements is inflated and the catheter is manipulated after the chosen one of the first and second balloon elements is inflated to urge an other one of the first and second balloon elements away from the chosen one of the first and second balloon elements before the other one of the first and second balloon elements is inflated.

16. The apparatus of claim 6 wherein at least a portion of a chosen one of the first and second balloon elements has at least one of a different size, shape, material, thickness, permeability, conductivity, and resilience from a corresponding portion of an other one of the first and second balloon elements.

17. The apparatus of claim 6 wherein the balloon elements are segments of a single balloon.

18. A method of exerting a radially outward force on a body lumen, the method comprising:
(a) providing a balloon catheter including first and second inflatable balloon elements, the first balloon element when inflated including a surface that defines a recess to receive at least a portion of the second balloon element, the first and second balloon elements being arranged sequentially relative to each other along the catheter;
(b) inserting the balloon catheter into the body lumen;
(c) positioning the balloon catheter within the body lumen such that at least one of the first and second balloon elements is located radially adjacent the stenosis; and
(d) expanding the at least one of the first and second balloon elements so as to exert a radially outward force on the body lumen.

19. The method of claim 18 wherein the surface of the first balloon element when inflated has a shape that generally conforms to a shape of an adjacent surface of the second balloon element when inflated.

20. The method of claim 19 wherein the surface of the first balloon element has a generally concave shape and the adjacent surface of the second balloon element has a generally convex shape.

21. The method of claim 18 wherein the body lumen is a lumen of a blood vessel.

22. The method of claim 18 wherein a portion of at least one of the first and second balloon elements overlaps a portion of another of the first and second balloon elements along a length of the catheter.

23. The method of claim 18 comprising:
(e) mounting the intraluminal prosthesis on the balloon catheter; and
(f) expanding the at least one of the first and second balloon elements so as to exert a radially outward force on the intraluminal prosthesis to expand the intraluminal prosthesis at a predetermined site in the body lumen.

24. The method of claim 23 wherein the intraluminal prosthesis includes at least one articulation point at which the intraluminal prosthesis can bend, the at least one articulation point being disposed radially adjacent an interface between the first and second balloon elements.

25. The method of claim 18 wherein the balloon catheter is used to dilate a stenosis in the body lumen, the method comprising:
(e) positioning the balloon catheter within the body lumen such that at least one of the first and second balloon elements is located radially adjacent the stenosis in a curved portion of the body lumen; and
(f) expanding the at least one of the first and second balloon elements so as to exert a radially outward force on the stenosis to dilate the stenosis.

26. The method of claim 18 wherein at least one of the first and second balloon elements is expanded asynchronously with expansion of an other one of the first and second balloon elements.

27. The method of claim 26 comprising:
(e) expanding a chosen one of the first and second balloon elements; and
(f) manipulating the catheter after the chosen one of the first and second balloon elements is expanded to urge an other one of the first and second balloon elements away from the chosen one of the first and second balloon elements before the other one of the first and second balloon elements is expanded.
